(19) United States
(12) Patent Application Publication
(10) Pub. No.: US 2018/0055797 A1
(43) Pub. Date: Mar. 1, 2018

(54) NON-RACEMIC KETONE SALTS FOR RAPID-ONSET NUTRITIONAL KETOSIS AND METABOLIC THERAPY

(71) Applicant: KetoneAid Inc., Falls Church, VA (US)

(72) Inventors: Frank Borges LLosa, Falls Church, VA (US); Stephen Zarpas, Washington, DC (US)

(73) Assignee: KetoneAid Inc., Falls Church, VA (US)

(21) Appl. No.: 15/690,893

(22) Filed: Aug. 30, 2017

Related U.S. Application Data
(60) Provisional application No. 62/381,567, filed on Aug. 31, 2016.

(51) Int. Cl. A61K 31/19; A61K 9/00; A23L 33/10

(52) U.S. Cl. CPC A61K 31/19; A23V 2002/00; A23L 33/10; A61K 9/0056

(57) ABSTRACT

A foodstuff can include sodium D-β-hydroxybutyrate, potassium D-β-hydroxybutyrate, and/or calcium D-β-hydroxybutyrate.

### CROSS REFERENCE TO RELATED APPLICATION

This application claims priority to U.S. provisional application 62/381,567 filed Aug. 31, 2016 which is hereby incorporated by reference.

### TECHNICAL FIELD

This invention generally relates to compositions and methods for producing near instant and/or therapeutic levels of nutritional ketosis, and in particular compositions and methods related to the right hand enantiomer in particular in either in its pure enantiomer form or enantiomerically enriched form of D-β-hydroxybutyrate salts for mitochondrial health, treating other conditions, and physical performance.

### BACKGROUND

DL-β-HB(Na+) salt has been used in the past, but its use has been limited due to limitations on sodium intake. A combination of other non-racemic D-β-hydroxybutyrate salts alongside non-racemic D-β-HB(Na+) such that none exceed their recognized daily metabolic limit, provides more rapid onset and sustained ketosis for therapeutic applications.

### SUMMARY

An aspect can include a foodstuff having sodium D-β-hydroxybutyrate, potassium D-β-hydroxybutyrate, and/or calcium D-β-hydroxybutyrate. In some embodiments, the ratio of sodium, potassium, and calcium D-β-hydroxybutyrate salts can be in a range of 1.75-3.5 parts sodium D-β-hydroxybutyrate, 2.0-3.5 parts potassium D-β-hydroxybutyrate, and 1.75-2.5 calcium D-β-hydroxybutyrate. In yet other embodiments, the D-β-hydroxybutyrate salt can be enantiomerically pure.

An aspect can include a mixture of enantiomerically enriched β-hydroxybutyrate salts having enantiomerically enriched sodium β-hydroxybutyrate and at least one additional enantiomerically enriched β-hydroxybutyrate salt. In some embodiments, the at least one additional enantiomerically enriched β-hydroxybutyrate salt can be potassium β-hydroxybutyrate and/or calcium β-hydroxybutyrate.

An aspect can include a foodstuff having limited racemic sodium β-hydroxybutyrate, racemic potassium β-hydroxybutyrate, and/or racemic calcium β-hydroxybutyrate such that the preponderance of the composition is non-racemic or enantiomerically enriched.

Other features and associated advantages will become apparent with reference to the following detailed description of specific embodiments in connection with the accompanying drawings.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a line-angle formula of sodium D-β-hydroxybutyrate.

FIG. 2 depicts an exemplary ketone blood concentration after ingestion of racemic 1,3-butanediol.

FIG. 3 depicts a ketone blood concentration after one person ingested (D) 1,3-butanediol, compared to the results of two people ingesting racemic 1,3-butanediol.

### DETAILED DESCRIPTION

A detailed explanation of the composition of matter and process according to preferred embodiments of the present invention are described below.

Ketosis is a fat-based metabolism wherein the body produces almost exclusively the enantiomer D-β-hydroxybutyrate. Though occurring in very small quantities and an intermediate metabolite, L-β-hydroxybutyrate must be distinguished from the D version and is only created and used in very small quantities inside the mitochondria and is never found naturally circulating through the blood in any measurable amounts, a state indicated by elevated levels of ketones in the blood and in which a person's body produces ketones for fueling metabolism rather than using primarily using dietary forms of glucose or metabolizing glycogen to make glucose. The ketogenic diet, which can initiate and maintain ketosis, was developed initially to treat pediatric refractory epilepsy. The original diet required ingesting calories primarily from fat, with a minimally sufficient amount of proteins to allow for growth and repair, and with a very restricted amount of carbohydrates. A typical diet would include a 4:1 ratio of fat to combined protein and carbohydrate (by weight). The ketogenic diet can allow one's body to consume fats for fuel rather than carbohydrates. Normally, the carbohydrates contained in food are stored as glycogen in the body and then, when needed, converted into glucose. Glucose is particularly important in fueling brain-function.

When a body lacks carbohydrates, the liver converts fat into fatty acids and further into ketone bodies. The ketone bodies are able to pass into the brain and replace glucose by up to 70% as the primary fuel substrate. An elevated level of ketone bodies in the blood, i.e. ketosis, has been shown to reduce the frequency of epileptic seizures. Ketosis has been shown to improve brain-function by providing a critical source of fuel to fuel starved cells due to a pathologically compromised inability to completely oxidize glucose. That pathologic inability is very likely at the root of many well-known neurodegenerative diseases such as Alzheimer's Disease, Parkinson's Disease and amyotrophic lateral sclerosis (ALS). The pathologic inability to process glucose is also very likely at the core of Traumatic Brain Injury (TBI).

In addition to improved brain-function, ketones can improve muscle performance, such as in endurance athletes, and muscle recovery that would be beneficial to all athletes, including sprinters. Skeletal muscles show a higher affinity for ketones and in particular the enantiomerically pure ketone body D-β-hydroxybutyrate (D-β-HB) over glucose. D-β-HB is thermodynamically more powerful than glucose. D-β-HB produces more ATP per unit volume oxygen than glucose. This is because the body can only store and convert about 100-minutes' worth of glycogen into useful glucose during extreme and prolonged exercise, such as in bicycle races and long-distance running. Athletes can train to extend their body's capacity, but there are limits. Moreover, a clear decline in glucose can be measured within about 16 minutes of physical exertion. Yet, with a second or alternative source of energy, from ketones, the body can continue to perform beyond the individual's capacity to utilize glucose. Further, studies have shown that ketones can improve endurance performance by as much as eight percent.

Achieving therapeutic levels of ketones in the blood can be difficult and/or problematic if using only racemic DL-β-hydroxybutyrate sodium. For example, US Pre-grant Patent Publication 2006/0280721 A1, which is incorporated in its entirety herein by reference, states that “[a]dministration of the sodium salt of these compounds is also unsuitable due to a potentially dangerous sodium overload that would accompany administration of therapeutically relevant amounts of these compounds.” (Desrochers et al. J. Nutr. Biochem. 1995, 6, 111-118)

A solution can be a mixture of such constituents whose individual drawbacks do not compound when mixed with other individual constituents. It can be shown that specific mixtures of three components can safely lead to therapeutic levels of ketones in the blood. For example, sodium, potassium, and calcium D-β-hydroxybutyrate salts.

To be clear, the chemical prefix “D” as used herein includes both enantiomerically enriched and enantiomerically pure versions, unless stated otherwise or used in a context that makes clear that only the pure version is intended. In some preferred embodiments, chiral salts of D-β-HB can be specifically combined for additional efficacy with reduced, or even without, undesirable negative side effects of each part by itself. Further, a combination of these (D) compounds can allow for much higher levels of ketones, limiting the risk of acute acidosis, salt overload, and gastrointestinal distress, at the highest doses.

There can be several ways to increase ketone levels. As shown above, however, there can be significant drawbacks and limitations to each. Additional methods and considerations are discussed below. Nevertheless, novel and specific combinations have been discovered that can balance limitations against each other with a resulting mix that is therapeutic.

Ketosis can be induced through eating a ketogenic diet, e.g., a diet of approximately 80% fat, 15% protein, and 5% carbohydrates. Such diets are difficult to maintain and are often found to be unpalatable. Ketogenic diets are not practical for the general population. Moreover, only the strictest diets can achieve up to about 3 mmol/L of ketones. Total caloric restriction or “starvation ketosis” for 10 days or more can achieve levels as high as 8 mmol which may be considered as the upper level of endogenous nutritional ketosis, but total caloric restriction is obviously not maintainable.

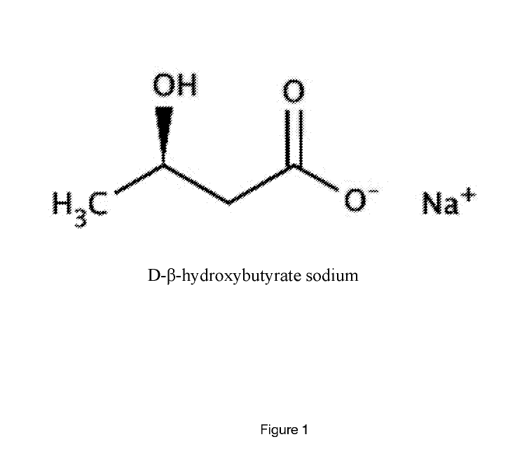

Several salts of D-β-hydroxybutyrate can be utilized to promote ketosis. For example, the sodium, potassium, and calcium salts are each useful and, within limitations, safely ingestible. The racemic sodium salt of β-hydroxybutyrate can be consumed to promote ketosis. However, regular consumption is limited by sodium's recommended dietary allowance (RDA) and daily upper limit, for example as set forth by the Food and Drug Administration. Most Americans currently consume roughly 50% in excess of the RDA for sodium. If a person's dietary sodium is limited to only sodium β-hydroxybutyrate, then that person would be limited to approximately 0.5 mmol/L of ketones by consuming racemic sodium β-hydroxybutyrate at about 100-200% of the RDA for sodium. (See U.S. Pat. No. 9,138,420, FIG. 1) That number falls considerably short of the 8 mmol upper level of nutritional ketosis.

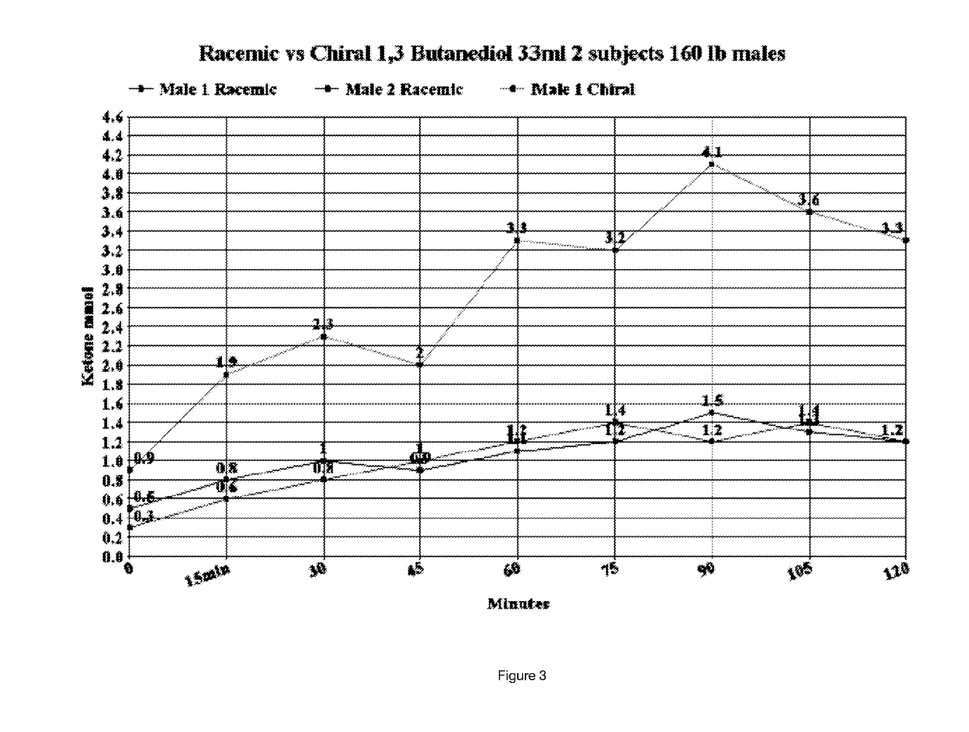

It should also be noted that only the D enantiomer is active in the body as a source of extracellular fuel that is then transported into the cells. Ketone blood level meters currently on the market only measure the blood level of the D enantiomer. Products containing racemic salts require two to three times the amount of sodium, calcium and potassium for an equal amount of D-β-hydroxybutyrate readings in the blood. A reason the potential levels of D-β-hydroxybutyrate in the blood is over double with chiral solutions is because the body has to waste energy and uses up some of the D-β-hydroxybutyrate to burn off the unnatural L-O-hydroxybutyrate. At the time of filing non-racemic salts were not available to us for testing; however, FIG. 3 shows the results from consuming 33 ml of (D) 1,3-butanediol, another compound that increases D-β-hydroxybutyrate in the blood, are over double that of 33 ml of racemic 1,3-butanediol.

In the paragraph below, ketone mmol levels are based on racemic salts.

Potassium β-hydroxybutyrate is another salt that can be consumed to promote ketosis, but as with sodium, potassium has a RDA and upper limit (UL) that limits consumption. By consuming racemic potassium β-hydroxybutyrate at about 100% of potassium's RDA, a person would be limited to reaching approximately 0.5 mmol/L. (See U.S. Pat. No. 9,138,420, FIG. 1) Further, the potassium salt can have an undesirable metallic taste that can limit people's willingness to consume this salt alone. Moreover, there are some medications that require strict limitations on potassium intake.

Consumption of calcium β-hydroxybutyrate salt is more limited than for the sodium and potassium salts. For example, calcium's RDA is approximately 1000 mg whereas sodium's RDA is over 2000 mg and potassium's RDA is nearly 5000 mg. Nevertheless, consumption of this salt, within limitations, can promote ketosis.

Lastly, (D)-β-hydroxybutyrate-D)-1,3-butanediol monoester can be an excellent, and previously unrivalled, driver of ketosis. But, the monoester is exorbitantly expensive. For example, a single dose of the monoester can cost upwards of $30,000 to produce.

Preferred embodiments utilize an optimized mix of one or more of the above ingredients (with the exception of (D)-β-hydroxybutyrate-(D)-1,3-butanediol monoester) to maximum ketone production, yet tailor the ingredients to account for recommended limitations, palatability, and deleterious side effects. Rapid inducement and maintenance of ketosis can be achieved, by utilizing certain optimized formulae, that in certain uses approaches the efficacy of (D)-β-hydroxybutyrate-, (D)-1,3-butanediol monoester at a tiny fraction of the cost of producing the monoester.

The salt mixture can be pure or, in a preferred embodiment, can be in a ratio by weight of 44% potassium salt, 32% sodium salt, and 24% calcium salt. This ratio, while not rigid, optimizes the salts according to their RDAs. The salts can be optimized according to individual consumer needs and/or FDA recommendations. The latter ratio can allow two times the dose of the former mixture while maintaining FDA recommendations for the salts.

Preferred compositions can be designed to reach target levels of 2.5-6 mmol/L of ketones in the blood. It has been shown that elite athletes can achieve an average of two percent and up to an eight percent improvement in performance with 5.6 mmol/L or higher. (See, for example, www.cell.com/cell-metabolism/fulltext/S1550-4131(16)30355-2) In a long term case study with an Alzheimer's patient, an obvious correlation in the mitigation of symptoms was made once blood levels reached 3-7 mmol/L.

D-β-HB is thermodynamically more energy dense than glucose. The oxidation of D-β-HB per unit volume of oxygen produces more energy than glucose. A direct correlation between the concentration in the blood to a minimum threshold and physical performance can be shown. Based on studies involving rats' hearts, Alzheimer's patients, and other studies, it may be shown ketone concentrations in the blood above various threshold minima can provide therapeutic effects for a variety of neurological conditions such as Alzheimer's, Parkinson's, ALS, Multiple Sclerosis, traumatic brain injury, epilepsy, and autism, as well as non-neurological conditions such as diabetes types I & II. For example, D-β-HB has been shown to act as a fuel substrate and substitute for glucose in diabetics as well as have hormone-like effects such as lowering of insulin levels.

While certain components within preferred embodiments have been investigated for their therapeutic value, it is important to note that each component within preferred embodiments is a foodstuff, not a pharmaceutical drug. Moreover, metabolic therapies have been investigated to provide mitochondria a source of energy needed to promote normal healthy metabolism in all people, healthy and otherwise. For example, U.S. Pat. No. 6,207,856, which is incorporated herein in its entirety, discusses administration of metabolic precursors in amounts sufficient to raise ketone bodies in blood. See Col. 5. The '856 patent explains that elevated levels of ketone body concentrations in the blood can result in not only maintenance of cell viability but improved cell function and growth beyond that of normal. The reference, however, fails to recognize or suggest present embodiments and, resultantly, fails to achieve the benefits of present embodiments. Several benefits of increased ketone bodies in healthy individuals can include nerve stimulant factors, i.e. nerve growth factors and factors capable of stimulating enhanced neuronal function, such as increased metabolic rate, retardation of degradation, and increased functional features such as axons and dendrites.

The rapidity of onset of available ketones in the blood can be of particular concern, for example to diabetics and/or athletes. Preferred embodiment can safely induce ketosis more rapidly than previously thought possible. For example, U.S. Pat. No. 9,138,420 shows that a peak concentration of D-β-HB produced by a combination of L,D-β-HB salt and MCT (medium chain triglycerides) oil required up to 2 hours. Further, subjects fasted prior to testing, which naturally increases ketone levels. For example, the subject who reached 1.3 mmol/L began the trial at 0.2 mmol/L. Thus, the net rise in ketones was approximately 1.1 mmol/L. The second subject began the trial at 0.9 thus the net rise in ketones was only about 1.65 mmol/L. What is more, each of the above trials required sodium consumption of approximately 2 grams. Reaching a target of 5-7 mmol/L in a 70 kg adult, using previous compositions would require approximately 16 g of sodium, far exceeding the daily recommended amount of 2.3 g per day. In addition, MCT oil is not tolerated well by the gut and can require an adaptation phase.

Therapies can be improved by limiting dietary carbohydrates and/or protein. Specifically, after administering or consuming an embodiment, blood levels of ketone bodies and/or cations of the salts can be measured. In a preferred method, one or more D-β-hydroxybutyrate salts can be administered. Then, the patient's blood levels can be measured for ketone bodies and/or salt levels. Based on the measurements, the dosage can be tuned to the particular patient. For example, if a patient's ketone levels are only reaching 0.3 mmol/L, then the dosage can be increased. As another example, the patient's ketone levels may be at 5.0 mmol/L but the patient's salt levels may be alarmingly high. In the latter example, the combination of constituents can be altered to reduce one particular salt or the entire dose can be reduced.

Nutritional ketosis has not previously been sustainable in different contexts. For example, metabolization of ketones can vary based on the metabolic rate of a particular individual. As another example, an athlete can burn a concentration of 6 mmol/L to less than 1 mmol/L in as little at 75 minutes of exertion. Prior thoughts have been to buffer the free acid with sodium salts. See, e.g., U.S. Pat. No. 9,138,420. But, this can cause harmful sodium overload and mineral imbalance, especially to achieve therapeutic levels of ketosis. Prior attempt have also failed to appreciate the importance of specific combinations that present embodiments include. For example, the '420 patent is directed to β-hydroxybutyrate in general as a compound and lists scores of β-hydroxybutyrate compounds as potential precursors, but fails to appreciate which compounds are efficacious or even safe (e.g. listing a lithium salt that can be dangerous). And further, it fails to appreciate the superiority of utilizing chiral compounds and mistakenly suggests that racemic compounds are as efficacious as enantiomerically enriched or pure compounds, which is contrary to our findings with racemic versus non-racemic compounds such as 1,3 butanediol. To address prior problems, preferred embodiments can increase ketone concentration in the blood more rapidly than previously thought possible to do safely. Indeed, present embodiments are a stark departure from previous paradigms and attempts to induce and maintain ketosis. For example, by using a sodium such as calcium or potassium salt of the non-racemic D-β-HB, the amount of salt and mineral imbalance can be cut by more than half, yet achieve improved results.

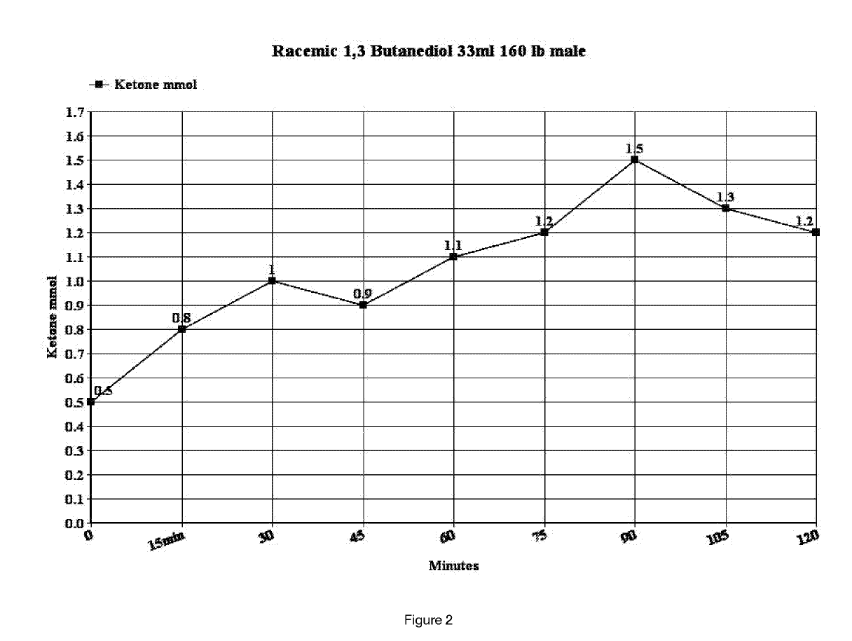

FIG. 2 depicts exemplary results from ingestion of 33 ml racemic 1,3-butanediol. As can be seen, racemic 1,3-butanediol alone achieved as much as 1 mmol/L over a 105-minute period. FIG. 3, on the other hand, shows that ingestion of (D) (as opposed to racemic) (D)-1,3-butanediol can have markedly improved efficacy in achieving ketosis. For example, as shown, the chiral form provided an increase in ketones of approximately 3.2 mmol/L whereas the racemic form provided approximately 0.9-1.0 mmol/L increases over pre consumption ketone levels.

As one skilled in the art will appreciate, embodiments of the present invention may be embodied as, among other things, a composition of matter and a method for making compositions of matter. Other embodiments are within the scope of the following claims. For example, while persons and patients are described herein, many advantages of embodiments can be provided to other animals, such as livestock, pets, horses, and work animals.

We claim:

1. A foodstuff comprising:
D-β-hydroxybutyrate salts.

2. The foodstuff of claim 1, wherein the D-β-hydroxybutyrate salt comprises Na+.

3. The foodstuff of claim 1, wherein the D-β-hydroxybutyrate salt comprises K+.

4. The foodstuff of claim 1, wherein the D-β-hydroxybutyrate salt comprises Ca+.

5. The foodstuff of claim 1, wherein the D-β-hydroxybutyrate salt comprises Na+ and Ca+.

6. The foodstuff of claim 1, wherein the D-β-hydroxybutyrate salt comprises Na+ and K+.

7. The foodstuff of claim 1, wherein the D-β-hydroxybutyrate salt is enantiomerically pure.

8. A foodstuff comprising:
sodium D-β-hydroxybutyrate;
potassium D-β-hydroxybutyrate; and
calcium D-β-hydroxybutyrate.

9. The foodstuff of claim 8, wherein the ratio of sodium D-β-hydroxybutyrate, the potassium D-β-hydroxybutyrate, and the calcium D-β-hydroxybutyrate is in a range of 1.75-3.5 parts sodium D-β-hydroxybutyrate, 2.0-3.5 parts potassium D-β-hydroxybutyrate, and 1.75-2.5 calcium D-β-hydroxybutyrate.

10. The foodstuff of claim 8, wherein the sodium D-β-hydroxybutyrate, the potassium D-β-hydroxybutyrate, and the calcium D-β-hydroxybutyrate are each enantiomerically pure.

11. A mixture of enantiomerically enriched β-hydroxybutyrate salts comprising:
enantiomerically enriched sodium β-hydroxybutyrate; and
at least one additional enantiomerically enriched β-hydroxybutyrate salt.

12. The mixture of claim 11, wherein at least one additional enantiomerically enriched β-hydroxybutyrate salt is potassium β-hydroxybutyrate.

13. The mixture of claim 11, wherein at least one additional enantiomerically enriched β-hydroxybutyrate salt is calcium β-hydroxybutyrate.
